(19) United States
(12) Patent Application Publication
(10) Pub. No.: US 2006/0282002 A1
(43) Pub. Date: Dec. 14, 2006

(54) Respiratory testing system

(75) Inventors: Jui-Chi Wang, Taipei (TW); Wen-Feng Yang, Hsinchu County (TW); Shiow-Chen Wang, Taipei (TW)

Correspondence Address: BAKER & MCKENZIE LLP, Pennzoil Place, South Tower, 711 Louisiana, Suite 3400, HOUSTON, TX, 77002-2716, US

(73) Assignee: TreatYou Medical Technology Co., Taipei City (TW)

(21) Appl. No.: 11/149,897

(22) Filed: Jun. 10, 2005

(51) Int. Cl. A61B 5/08

(52) U.S. Cl. 600/538

(57) ABSTRACT

A respiratory testing system comprises a fluid chamber enclosing a fluid medium and having a flexible membrane deflectable by the application of a respiratory gas flow, a pressure sensor configured to sense a pressure variation of the fluid medium in response to a deflection of the flexible membrane, and a processing circuit configured to compute a respiratory flow data from a pressure variation of the fluid medium. The respiratory testing system may additionally include a breathing exercise unit configurable to execute a software program responsive to an input of the respiratory flow data.

### FIELD OF THE INVENTION

The present invention generally relates to respiratory measurements, and more particularly to the construction of a respiratory testing system that can determine respiratory flow data and provide breathing exercise functions.

### DESCRIPTION OF THE RELATED ART

Spirometry tests are conventionally conducted in the prevention, diagnosis, observation and therapy of pulmonary diseases such as asthma, bronchitis, mucoviscidosis, etc. During a spirometry test, a patient induces an inspiratory or expiratory gas flow through a gas flow tube. A flow measurement mechanism measures the gas flow rate, which then may be shown on a display screen connected to the measurement mechanism.

Various types of flow measurement mechanism are implemented in existing spirometer systems. In one traditional spirometer system, a rotating fan is installed at an end of the gas flow tube. The inspiratory or expiratory gas flow induced by a user or patient through the gas flow tube causes the fan to rotate. The respiratory flow rate is inferred by counting the number of revolutions per time unit of the fan.

Another spirometer system known in the prior art uses pressure signals provided by a pressure sensor inside the gas flow tube to determine respiratory flow data. U.S. Pat. Nos. 5,111,827; 5,816,246; 6,019,731; and 6,322,519, the disclosures of which are incorporated herein by reference, describe exemplary constructions of a spirometer system based on a pressure sensor implemented as a flow measurement mechanism. The respiratory gas flow created along the gas flow tube modifies its inner pressure, which is sensed by the pressure sensor that issues an analog signal being converted into a digital form before it is inputted to a processor unit. Respiratory flow data then are computed within the processor unit, and displayed on a monitor screen.

A study of existing products available on the market reveals that improvements can be brought to the spirometer system of the prior art. For example, the spirometer functions are conventionally limited to the provision of respiratory flow data, and fail to provide useful evaluation and testing of the respiratory functions. Furthermore, the conventional construction based on a pressure sensor measurement usually puts the pressure sensor in direct contact with the respiratory gas flow induced by the user, which can result in a contamination of the sensor part by dirt or germs conveyed along with the respiratory gas flow. An air-permeable filter may be used to remedy this problem, but it adversely increases the manufacturing cost.

Therefore, there is presently a need for a spirometer system that can overcome the foregoing technical problems, and further can be manufactured with an economical cost.

### SUMMARY OF THE INVENTION

In one embodiment, a respiratory testing system comprises a fluid chamber enclosing a fluid medium, and a processing circuit configured to determine respiratory flow data from a pressure variation of the fluid medium being generated in response to the application of a respiratory gas flow.

In some embodiments, the fluid medium includes air. In some embodiments, the fluid chamber includes a flexible membrane deflectable by the application of a respiratory gas flow. In some embodiments, one side of the flexible membrane is in contact with the fluid medium. In some embodiments, the processing circuit is configured to compute respiratory flow data according to a proportional relation between a pressure of the fluid medium and a respiratory flow rate.

In some variations, the respiratory testing system includes a breathing exercise unit. In some embodiments, the breathing exercise unit is operable to execute a software program configured to respond to an input of respiratory flow data.

This application also describes a method of evaluating a respiratory flow through a testing system. In some embodiments, the method comprises enclosing a fluid medium inside the testing system, sensing a pressure variation of the fluid medium in response to a respiratory gas flow applied against the fluid medium, and determining a respiratory flow rate according to a pressure variation of the fluid medium.

In some embodiments, determining a respiratory flow rate according to a pressure variation of the fluid medium includes computing a proportional relation between the pressure of the fluid medium and the respiratory flow rate. In other embodiments, the method further comprises executing a breathing exercise program configured to respond to an input of respiratory airflow data.

The foregoing is a summary and shall not be construed to limit the scope of the claims. The operations and structures disclosed herein may be implemented in a number of ways, and such changes and modifications may be made without departing from this invention and its broader aspects. Other aspects, inventive features, and advantages of the invention, as defined solely by the claims, are described in the non-limiting detailed description set forth below.

### BRIEF DESCRIPTION OF THE DRAWINGS

FIG. 1 is a general view of a respiratory testing system according to an embodiment of the invention;

FIG. 2 is a cross-sectional view of a portion of the gas flow tube implemented in a respiratory testing system according to an embodiment of the invention;

FIG. 3 is a schematic view of a processing circuit implementation in a respiratory testing system according to an embodiment of the invention; and

FIG. 4 is a flowchart illustrating an execution sequence of a breath exercise program within a respiratory testing system according to an embodiment of the invention.

### DETAILED DESCRIPTION OF THE EMBODIMENTS

This application describes a respiratory testing system that can determine respiratory flow data and test pulmonary functions by simulating respiratory exercises.

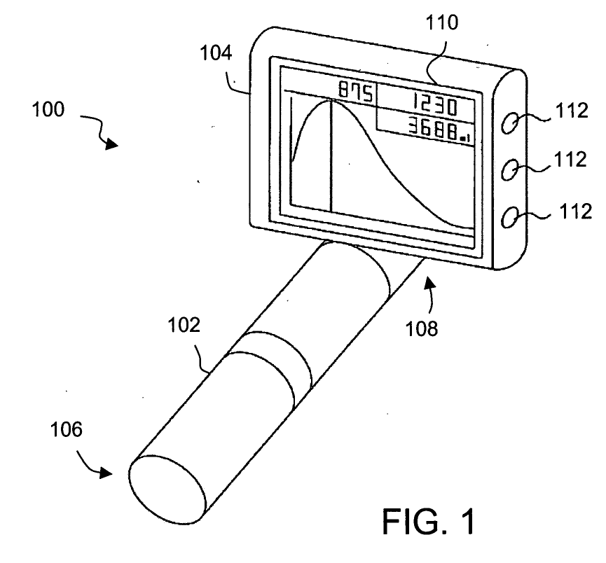

FIG. 1 is a schematic view of a respiratory testing system according to an embodiment of the invention. Reference number 100 generally designates a respiratory testing system, which includes a gas flow tube 102 and a control body 104. The gas flow tube 102 terminates in a first end 106 through which a user breaths in to create a gas flow inside the tube 102, while a second end 108 of the gas flow tube 102 is open to evacuate the gas flow inside the gas flow tube 102 to the outside.

The control body 104 encloses an electric processing circuit of the respiratory testing system. On the control body 104 are arranged a display screen 110 implementing a user graphic interface, and functional buttons 112 through which the user can control the operation of the system 100. In an embodiment, the display screen 110 may be a touch panel screen, and functional buttons 112 may include an on/off-switch button to power on the system, a reset button to reinitialize the system, and any control buttons or key pads configured to command functions provided in the system. Though the embodiment of FIG. 1 shows the control body 104 being mounted to the gas flow tube 102, other embodiments may separate the control body from the gas flow tube with a flexible signal cable electrically connecting these two elements. Alternatively, the gas flow tube 102 and the control body 104 may be formed as one single body. Additionally, the functional buttons 112 may be arranged on the gas flow tube 112 rather than on the control body 104.

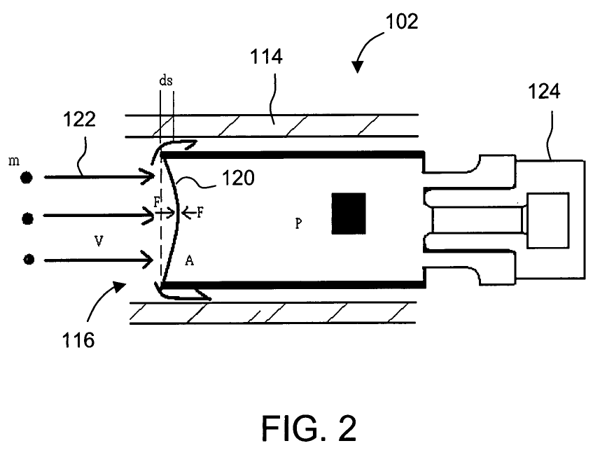

FIG. 2 is a cross-sectional view of a portion of the gas flow tube implemented in a respiratory testing system according to an embodiment of the invention. Any suitable materials such as plastics or the like can be used to form the gas flow tube 102, which has a wall 114 defining an inner cavity 116 in which is mounted a chamber enclosing a fluid medium, such as air chamber 118. The air chamber 118 does not occupy completely the inner cavity 116, and gaps are left between the air chamber 118 and the wall 114 to allow a created gas flow to travel along the tube 102. The air chamber 118 has a membrane 120 that extends transversal to the gas flow 122, and is in contact with the air enclosed in the air chamber 118. A pressure sensor 124 is connected to the air chamber 118 to sense a pressure variation within the air chamber 118. Analog signals issued by the pressure sensor 124 are wired to a processing circuit inside the control body 104.

Referring to FIGS. 1 and 2, subject to a gas flow created inside the gas flow tube 102 when, for example, a user exhales gas through the tube end 106, the membrane 120 deforms and causes a pressure variation inside the air chamber 118. The pressure sensor 124 senses this pressure variation, which is used to determine respiratory flow data. Since the sensitive part of the pressure sensor 124 communicates with an enclosed air, damageable contaminations of the pressure sensor 124 can be prevented.

In conjunction with FIG. 2, the following description illustrates an exemplary method of determining a gas flow rate inside a tube from a pressure variation of an enclosed fluid medium according to an embodiment of the invention. When the deformed membrane 120 is in a state of force equilibrium, equilibrium force F can be formulated by the two following relations applied at each side of the membrane 120:
F=Σm·dV/dt; (1)

wherein F is the equilibrium force, m is the punctual mass of one particle transported by the gas flow 122, V is a particle velocity, and dV/dt is the time derivation of particle velocity V; and
F=P·A+F_{membrane}=P·A+k·ds; (2)

wherein P is the pressure inside the air chamber 118, A is a transversal section area of the air chamber 118, F_{membrane} is the reaction force produced by the membrane 120, k is the elasticity coefficient of the membrane 120, and ds is the amount of deflection of the membrane 120.

Since the constant k is substantially small (very close to 0), the term (k·ds) can be neglected and the relation (2) can be simplified to the following:
F=P·A; (2′)

On the other hand, a gas flow rate Q through the gas flow tube 102 can be expressed by the following relation:
Q=A_{tube}·∫dV/dt·dt; (3)

wherein Q is a gas flow rate, and A_{tube} is a transversal section area of the gas flow tube 102.

Combining the relations (1), (2′) and (3) results in the following relation in which the airflow rate Q is proportional to the pressure inside the air chamber:
Q=C·P; (4)

wherein C is a constant to be determined according to constant parameters such as the section area of the tube and the section area of the air chamber.

The equation (4) establishes a relation between a gas flow through the tube 102 and the pressure within the air chamber 118. This relation can be used to compute respiratory flow data. Additionally, depending on the pressure P inside the air chamber 118, the differences between a user's inspiratory and expiratory gas flow can be determined and trained.

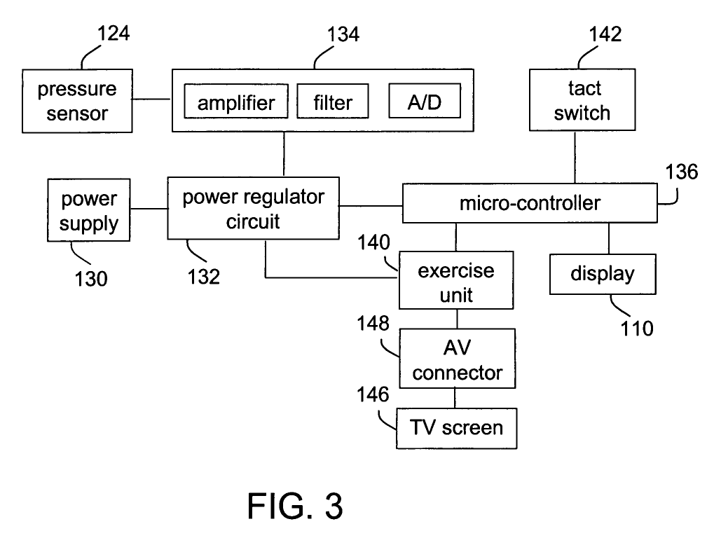

FIG. 3 is a schematic view of a processing circuit implementation in a respiratory testing system according to an embodiment of the invention. Referring to FIG. 2 and FIG. 3, a power supply 130 provides the necessary power via a power regulator circuit 132 to the processing circuit. One or more tact switches 142 are triggered when the user actuates the functional buttons 112. When a user inhales or exhales gas through the gas flow tube 102, a pressure variation inside the air chamber 118 is reflected by an analog signal outputted from the pressure sensor 124. The analog signal from the pressure sensor 124 is processed via a converter circuit 134, including components such as an amplifier, filter and analog-to-digital (A/D) converter, operable to convert the analog signal to a suitable digital form.

A micro-controller 136 receives a digital signal reflecting the pressure inside the air chamber 118, and computes respiratory flow data such as a peak flow rate, a mean flow rate, forced expiratory volumes in successive breaths, or the like. The respiratory flow data can be shown on the display screen 110 in diverse graphic forms such as histograms, time evolution curves, numerical values or the like. In addition, the computed respiratory flow data may be further processed through a software program for additional evaluation and/or testing purposes.

Referring again to FIG. 3, a breathing exercise unit 140 may be incorporated in the respiratory testing system according to one embodiment of the invention. The breathing exercise unit 140 can include elements such as a read-only-memory resource, a processor unit and like components necessary to execute one or more software programs (also called “exercise program” hereafter) loadable in the system and configured to simulate a virtual environment in which the user can conduct breathing exercises. The selection, control and operation of a specific program by a user can be conducted through one or more functional buttons 112. The user can follow the execution of one exercise program on the display screen 110 incorporated in the system, or alternatively via a separate monitor device such as a television screen 146 being connected to the system via one audio-video (AV) terminal connector 148.

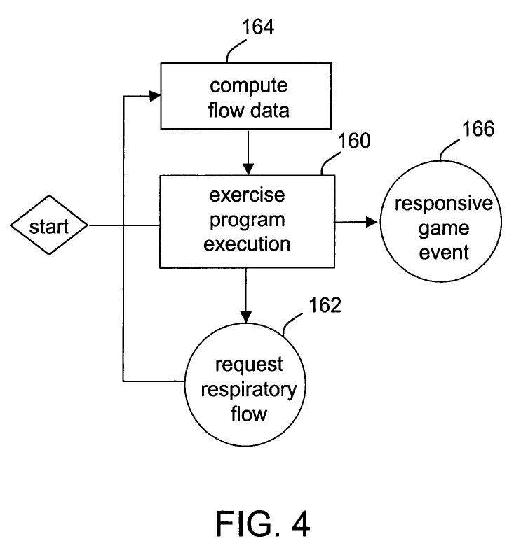

FIG. 4 is a flowchart illustrating an execution sequence of a breathing exercise program in a respiratory testing system according to an embodiment of the invention. In this embodiment, at least one exercise program simulates a game scheme that responds to inputs of respiratory flow data received from the micro-controller. The game scheme can be set according to any arbitrary set of rules predefined by a programmer. Initially, a breathing exercise program is executed by a processor of the exercise unit (160). During its execution, the exercise program may request the user to produce a respiratory flow through the gas flow tube of the respiratory testing system (162). Respiratory flow data are computed from a respiratory gas flow induced by the user (164), and inputted to the exercise program for evaluation. According to the result of this evaluation, the program execution then may produce a responsive game event (166). The production of respiratory gas flows may be repeatedly requested to the user until the evaluation meets a target value, which may correspond to another responsive game event. The system thus can test the user's pulmonary functions by conducting respiratory exercises through a game simulation.

The respiratory testing system according to this invention can be embodied in any forms. In a portable form, the respiratory testing system can be implemented in a hand-held appliance that can be conveniently manipulated by a user. Alternatively, the respiratory testing system can be integrated in a health testing station installed in a health or medical center.

Furthermore, many variations of the respiratory testing system can be implemented without departing from the inventive features described herein. For example, a mechanical assembly of movable parts (such as a sliding piston assembly) may substitute for the flexible membrane to cause a pressure variation inside the fluid chamber in response to the application of a respiratory flow.

In other variations, the fluid chamber may contain fluid elements other than air chosen with respect to their density so as to provide a pressure variation that adequately reflects the respiratory flow rate.

Realizations in accordance with the present invention therefore have been described in the context of particular embodiments. These embodiments are meant to be illustrative and not limiting. Many variations, modifications, additions, and improvements are possible. Accordingly, plural instances may be provided for components described herein as a single instance. Structures and functionality presented as discrete components in the exemplary configurations may be implemented as a combined structure or component. These and other variations, modifications, additions, and improvements may fall within the scope of the invention as defined in the claims that follow.

What is claimed is:

1. A respiratory testing system, comprising:
a fluid chamber, enclosing a fluid medium and having a flexible membrane deflectable by the application of a respiratory gas flow;
a pressure sensor configured to sense a pressure variation of the fluid medium in response to a deflection of the flexible membrane; and
a processing circuit configured to compute a respiratory flow data from a pressure variation of the fluid medium.

2. The system according to claim 1, wherein the fluid medium includes air.

3. The system according to claim 1, wherein one side of the flexible membrane is in contact with the fluid medium.

4. The system according to claim 1, wherein the flexible membrane lies substantially transversal to the respiratory gas flow.

5. The system according to claim 1, wherein the fluid chamber is mounted within a gas flow tube.

6. The system according to claim 1, wherein the processing circuit is configured to compute the respiratory flow data according to a proportional relation between a pressure of the fluid medium and a respiratory flow rate.

7. The system according to claim 1, further including a breathing exercise unit.

8. The system according to claim 7, wherein the breathing exercise unit is operable to execute a software program configured to respond to an input of respiratory flow data.

9. A method of evaluating a respiratory flow through a testing system, the method comprising:
enclosing a fluid medium inside the testing system;
sensing a pressure variation of the fluid medium in response to a respiratory gas flow applied against the fluid medium; and
determining a respiratory flow rate according to a pressure variation of the fluid medium.

10. The method according to claim 9, wherein determining a respiratory flow rate according to a pressure variation of the fluid medium includes computing a proportional relation between the pressure of the fluid medium and the respiratory flow rate.

11. The method according to claim 9, wherein the fluid medium includes air.

12. The method according to claim 9, further including executing a breathing exercise program configured to respond to an input of the respiratory airflow data.

13. A respiratory testing system comprising:
a fluid chamber enclosing a fluid medium therein; and
a processing circuit configured to determine a respiratory flow data from a pressure variation of the fluid medium, the pressure variation being generated in response to the application of a respiratory gas flow to the respiratory testing system.

14. The system according to claim 13, wherein the fluid chamber includes a flexible membrane deflectable by the application of the respiratory gas flow.

15. The system according to claim 14, wherein one side of the flexible membrane is in contact with the fluid medium.

16. The system according to claim 14, wherein the flexible membrane lies substantially transversal to the respiratory gas flow.

17. The system according to claim 13, wherein the fluid medium includes air.

18. The system according to claim 13, wherein the fluid chamber is mounted within a gas flow tube.

19. The system according to claim 13, wherein the processing circuit is configured to compute the respiratory flow data according to a proportional relation between a pressure of the fluid medium and a respiratory flow rate.

20. The system according to claim 13, further including a breathing exercise unit.

21. The system according to claim 20, wherein the breathing exercise unit is operable to execute a software program configured to respond to an input of the respiratory flow data.
